SEQUENCE LISTING (37 sequences)

SEQ ID NO: 1 - DNA, 38 bp, Artificial Sequence
  modified_base (6)..(10): ompA 5′-primer   “n” refers to inosine.
cgaggnnnnngactacccaaaccttcaacgacacctcg

SEQ ID NO: 2 - DNA, 38 bp, Artificial Sequence
  modified_base (6)..(10): ompA 3′-primer “n” refers to inosine.
cgcatnnnnncaagccaagaccgcaagtgaataatgcg

SEQ ID NO: 3 - DNA, 38 bp, Artificial Sequence
  modified_base (6)..(10): porA 5′-primer “n” refers to inosine.
ccaaannnnnttacagactggcggcggtttcgttttgg

SEQ ID NO: 4 - DNA, 35 bp, Artificial Sequence
  modified_base (6)..(10): porA 3′-primer “n” refers to inosine.
caagcnnnnngtcgactgcacacccgaacagcttg

SEQ ID NO: 5 - DNA, 36 bp, Artificial Sequence
  modified_base (6)..(10): btub1 5′-primer “n” refers to inosine.
cagctnnnnncctcgatgtcatccgtaaggaagctg

SEQ ID NO: 6 - DNA, 39 bp, Artificial Sequence
  modified_base (6)..(10): btub1 3′-primer   “n” refers to inosine.
gtcagnnnnngagcttacgaaggtcggagttgagctgac

SEQ ID NO: 7 - DNA, 47 bp, Artificial Sequence
  modified_base (6)..(10): gyrA 5′-primer   “n” refers to inosine.
catctnnnnntgccaatcctaagataaattccaaaccagaagagatg

SEQ ID NO: 8 - DNA, 41 bp, Artificial Sequence
  modified_base (6)..(10): gyrA 3′-primer   “n” refers to inosine.
gaccannnnncctagctccttataagcttgaactgctggtc

SEQ ID NO: 9 - DNA, 41 bp, Artificial Sequence
  modified_base (6)..(10): gap 5′-primer  “n” refers to inosine.
gatatnnnnncgcccgtcaaactatgggagctggtaatatc

SEQ ID NO: 10 - DNA, 46 bp, Artificial Sequence
  modified_base (6)..(10): gap 3′-primer   “n” refers to inosine.
gctttnnnnnctgagtttcctcattcggagatcaacggattaaagc

SEQ ID NO: 11 - DNA, 41 bp, Artificial Sequence
  modified_base (6)..(10): gap 5′-primer  “n” refers to inosine.
catgtnnnnnggatgaacgctggctgtgtgcctaatacatg

SEQ ID NO: 12 - DNA, 39 bp, Artificial Sequence
  modified_base (6)..(10): gap 3′-primer  “n” refers to inosine.
gactgnnnnncatcgctttctgacaaggtaccgtcagtc

SEQ ID NO: 13 - DNA, 38 bp, Artificial Sequence
  modified_base (6)..(10): 16s 5′-primer  “n” refers to inosine.
cactcnnnnnggagtttttgtgcacgctcggttgagtg

SEQ ID NO: 14 - DNA, 32 bp, Artificial Sequence
  modified_base (6)..(10): 16s 3′-primer  “n” refers to inosine.
cgtgannnnncaacggtctgggcagattcacg

SEQ ID NO: 15 - DNA, 36 bp, Artificial Sequence
  modified_base (6)..(10): phr1 5′-primer  “n” refers to inosine.
cgcaannnnncatcgaatctttgaacgcacattgcg

SEQ ID NO: 16 - DNA, 34 bp, Artificial Sequence
  modified_base (6)..(10): phr1 3′-primer  “n” refers to inosine.
gcggtnnnnngatatacgtggtggacgttaccgc

SEQ ID NO: 17 - DNA, 34 bp, Artificial Sequence
  modified_base (6)..(10): glyC 5′-primer  “n” refers to inosine.
ccaggnnnnngtcaacgaccatattcacgcctgg

SEQ ID NO: 18 - DNA, 38 bp, Artificial Sequence
  modified_base (6)..(10): glyC 3′-primer  “n” refers to inosine.
gagatnnnnncagacggagccgttggtgataagatctc

SEQ ID NO: 19 - DNA, 41 bp, Artificial Sequence
  modified_base (6)..(10): glyC 5′-primer  “n” refers to inosine.
cttgannnnngatgtttgcttggtcgttcctggtcctcaag

SEQ ID NO: 20 - DNA, 33 bp, Artificial Sequence
  modified_base (6)..(10): glyC 3′-primer  “n” refers to inosine.
catgannnnnccgtcgggactgaacgtctcatg

SEQ ID NO: 21 - DNA, 38 bp, Artificial Sequence
  modified_base (6)..(10): gap 5′-primer  “n” refers to inosine.
gcaagnnnnngtccatttcaacaagcacgcaaacttgc

SEQ ID NO: 22 - DNA, 48 bp, Artificial Sequence
  modified_base (6)..(10): gap 3′-primer  “n” refers to inosine.
cacctnnnnntggagcattaatttggctatcatctttttgaataggtg

SEQ ID NO: 23 - DNA, 42 bp, Artificial Sequence
  modified_base (6)..(10): p47 5′-primer  “n” refers to inosine.
gtatgnnnnngtgcgtactcggagcttgcagagaagacatac

SEQ ID NO: 24 - DNA, 48 bp, Artificial Sequence
  modified_base (6)..(10): p47 3′-primer  “n” refers to inosine.
cgaggnnnnngggctgcaattctttgttcttcgagttttcgtgcctcg

SEQ ID NO: 25 - DNA, 43 bp, Artificial Sequence
  modified_base (6)..(10): p27 5′-primer  “n” refers to inosine.
gatggnnnnnggtcgcattatagccaaagttaccgctaccatc

SEQ ID NO: 26 - DNA, 45 bp, Artificial Sequence
  modified_base (6)..(10): p27 3′-primer  “n” refers to inosine.
ctgaannnnntcaaatccgtcagcaaattgaagattcaacttcag

SEQ ID NO: 27 - DNA, 31 bp, Artificial Sequence
  modified_base (6)..(10): MTHFR 5′-primer  “n” refers to inosine.
atcttnnnnntgctgttggaaggtgcaagat

SEQ ID NO: 28 - DNA, 28 bp, Artificial Sequence
  modified_base (6)..(10): MTHFR WILD 3′-primer  “n” refers to inosine.
ccgatnnnnngcgtgatgatgaaatcgg

SEQ ID NO: 29 - DNA, 28 bp, Artificial Sequence
  modified_base (6)..(10): MTHFR MUTANT 3′-primer  “n” refers to inosine.
tcgatnnnnngcgtgatgatgaaatcga

SEQ ID NO: 30 - DNA, 30 bp, Artificial Sequence
  modified_base (6)..(10): BRAF 5′-primer  “n” refers to inosine.
caatgnnnnngaatatctgggcctacattg

SEQ ID NO: 31 - DNA, 28 bp, Artificial Sequence
  modified_base (6)..(10): BRAF WILD 3′-primer  “n” refers to inosine.
tgaaannnnncactccatcgagatttca

SEQ ID NO: 32 - DNA, 28 bp, Artificial Sequence
  modified_base (6)..(10): BRAF MUTANT 3′-primer  “n” refers to inosine.
agaaannnnncactccatcgagatttct

SEQ ID NO: 33 - DNA, 28 bp, Artificial Sequence
  modified_base (6)..(10): APC 5′-primer  “n” refers to inosine.
gaggtnnnnnccacacagaactaacctc

SEQ ID NO: 34 - DNA, 28 bp, Artificial Sequence
  modified_base (6)..(10): APC WILD 3′-primer  “n” refers to inosine.
agtttnnnnnttatgagaaaagcaaact

SEQ ID NO: 35 - DNA, 28 bp, Artificial Sequence
  modified_base (6)..(10): APC MUANT 3′-primer  “n” refers to inosine.
ggtttnnnnnttatgagaaaagcaaacc

SEQ ID NO: 36 - DNA, 44 bp, Artificial Sequence
  modified_base (6)..(10): GAPDG 5′-primer  “n” refers to inosine.
ggacannnnncacaagtatcactaagctcgctttcttgctgtcc

SEQ ID NO: 37 - DNA, 38 bp, Artificial Sequence; identical to SEQ ID NO: 3
  modified_base (6)..(10): GAPDH 3′-primer  “n” refers to inosine.

(19) United States
(12) Patent Application Publication
(10) Pub. No.: US 2018/0073082 A1
(43) Pub. Date: Mar. 15, 2018

(54) DUMBBELL-STRUCTURE OLIGONUCLEOTIDE, NUCLEIC ACID AMPLIFICATION PRIMER COMPRISING SAME, AND NUCLEIC ACID AMPLIFICATION METHOD USING SAME

(71) Applicant: DIOGENE CO., LTD, Seoul (KR)

(72) Inventors: Seong-Sik LIM, Namyangju-si (KR); Yeon-Soo KIM, Namyangju-si (KR)

(21) Appl. No.: 15/509,179

(22) PCT Filed: Feb. 25, 2015
(86) PCT No.: PCT/KR2015/001835

§ 371 Date: Mar. 6, 2017

(51) Int. Cl. C12Q 1/68; C12Q 1/70

(52) U.S. Cl. CPC C12Q 1/6886; C12Q 1/705; C12Q 1/689; C12Q 1/6883

(57) ABSTRACT

The present invention relates to a dumbbell-structure oligonucleotide (DSO), a nucleic acid amplification primer comprising the same, and a nucleic acid amplification method using the same and, more specifically, to a method for multi-gene amplification and single-nucleotide polymorphism analysis using a dumbbell-structure oligonucleotide capable of excluding non-specific amplification products prior to binding to a template in a first cycle in performing a polymerase chain reaction. The present invention suppresses undesired amplification products at room temperature using a dumbbell structure oligonucleotide (DSO) generated by adding any nucleotide sequence designed to allow the 5′-terminal oligonucleotide and the 3′-terminal oligonucleotide to complimentarily bind to each other, a 3′-terminal template dependent specific nucleotide sequence, and a universal nucleotide pair for linking the two nucleotide sequences, prior to binding to a template in every first cycle at the time of the polymerase chain reaction (PCR), and as a result, efficiently increases sensitivity and specificity through the reduction in non-specific amplification products, thereby achieving the innovation of the gene amplification method.

### CROSS-REFERENCE TO RELATED APPLICATIONS

This patent application is a national-stage application of International Patent Application No. PCT/KR2015/001835 filed on Feb. 25, 2015.

### TECHNICAL FIELD

The present invention relates to a dumbbell-structure oligonucleotide (DSO), a nucleic acid amplification primer comprising the same, and a nucleic acid amplification method using the same, and more specifically, to a method for multi-gene amplification and single nucleotide polymorphism analysis using a dumbbell-structure oligonucleotide, capable of excluding non-specific amplification products prior to binding to a template in each first cycle in performing a polymerase chain reaction.

### DISCUSSION OF RELATED ART

For obtaining genetic samples, researchers generally use polymerase chain reactions using DNA polymerase. Oligonucleotide used in the polymerase chain reaction is designed to bind to the opposite strand of a template DNA. This method has an advantage in that only the desired site of the target gene may be accurately amplified by arbitrarily controlling and designing the length and the base sequence of the oligonucleotide capable of binding to the template DNA. However, if the number of target genes to be amplified is large, the same operation should be repeatedly performed because only one target gene may be amplified in a single reaction.

In order to solve these drawbacks, efforts have been actively made to develop a number of methods for performing the polymerase chain reaction by mixing two or more kinds of template genes and primers corresponding to each template gene into one. Further, many methods have been developed to increase the binding specificity of primers and to enable amplification of resulting products. For examples, there are a touchdown polymerase chain reaction (PCR) (Don et al., 1991), a hot start PCR (DAquila et al., 1991), a nested PCR (Mullis and Faloona, 1987), and a booster PCR (Ruano et al., 1989). Still another approaches increase the specificity of the PCR using a variety of enhancer compounds, e.g., chemical compounds that increase an effective binding reaction temperature, DNA binding proteins, commercially available reactants. However, it is not possible to derive successful results from all PCR methods. Testing these additives takes a lot of time and effort under various binding temperature conditions. Although the approaches described above contribute somewhat to increasing primer annealing specificity, the approaches are not a fundamental solution to the problems resulting from the primers used for the PCR amplification, such as non-specific products and high background. Further, the number of genes that may be successfully amplified at a time is only three and four. There were unsolved disadvantages such as competition or interference effects between the genes, and amplification of nonspecific products causes the methods to be impractical.

In order to perform multiple polymerase chain reactions and allele-specific PCRs, it is necessary to optimize the conditions of the target template gene, which requires a lot of time, effort, and sample consumption. Such optimized conditions are not applied to other genes. In order to address these issues, various methods have been developed. For examples, there are a linker polymerase chain reaction (linker PCR) and a ligation mediated polymerase chain reaction (Ligation Mediated PCR) (Journal of Clinical Microbiology, 43 (11): 5622-5627, 2005). In the linker polymerase chain reaction, cross-contamination, however, is a serious problem because it may occur due to the characteristics of the experiment in which a part of the product reacted in the first tube is transferred to the second tube. In the ligation polymerase chain reaction, researchers undergo difficulties due to complex experimental methods using various kinds of enzymes. Thus, only some use such experimental techniques.

Accordingly, it has been required to develop techniques capable of performing inexpensively and simply gene amplification. In order to meet such demand, the technique developed simply controls only primer to disable the amplification until the PCR reaction temperature is suitable. An example of such a method is the invention disclosed in Korean Patent No. 649165. In this technique, an extra regulator site is additionally inserted in an initial primer. This regulator site is a polydeoxyinosine linker, and the inosine constituting the regulator site is a universal base having a lower Tₘ value than the general nucleotides G, A, T and C constituting a typical primer. Thus, at a certain temperature, the polydeoxyinosine linker forms a “bubble-like structure” to block a nonspecific binding of the primer to the template, thereby serving to suppress non-specific amplification of the PCR. Although such technique is somewhat inexpensive to implement in comparison with the prior art described above, there is an inconvenience that the temperature of the binding step in the first cycle (the PCR reaction temperature) and the temperature of the binding step in the second cycle should be different from each other in the real PCR. This is because the second PCR cycle to the sequence additionally inserted into the primer may participate in priming. Of course, this “application of different temperatures” is not necessarily required, but it may be necessary to apply different temperatures for efficient PCR. Further, a pre-selection arbitrary nucleotide sequence at the 5′-terminal site should be added in the above-mentioned technique, and there has been a restriction that it should not be complementary to any position of the template. This leads to additional inconvenience, and furthermore, if all the gene sequences of the template are unknown, it is uncertain to succeed in the technique's application. Therefore, it may be necessary to develop a new method which is cheaper and easier to implement than the prior art. Such suppression techniques of non-specific amplification are surely important in all PCRs and are more important in the PCRs used in the field of diagnosis, e.g., especially genetic tests and disease tests.

<Prior Art Document> (Patent Document 1) KR10-0649165 B

### SUMMARY

Thus, the present invention aims to address the problems of the prior art and the technical objectives required from the past.

The object of the present invention is to provide a PCR primer capable of suppressing non-specific amplification and a PCR method using the primer which suppresses unintended PCR amplification at room temperature to perform a hot-start PCR, and further, increases amplification from the PCR product in comparison with amplification from the initial template upon PCR amplification.

Accordingly, the present inventors have made extensive efforts to develop a method capable of amplifying a plurality of genes by only a single polymerase chain reaction. As a result, when preparing the primer of the gene base sequences to be amplified, upon using the primer comprising dumbbell structure oligonucleotide (DSO) to which the arbitrary base sequence complementary to the 3′-terminal of 3 bp to 5 bp at the 5′-terminal to form a dumbbell structure, and the universal base pairs of 3 bp to 5 bp connecting two sites are added, it was confirmed that a plurality of different genes may be rapidly and precisely amplified simultaneously by one polymerase chain reaction, and thus the present invention has been completed.

As a result, the main object of the present invention is to provide a method for amplifying the gene that may be present in all of samples only by the single polymerase chain reaction using a primer in which the universal base pairs of 3 bp to 5 bp connecting a template-specific base sequence and complementary arbitrary base sequence of the 3′-terminal of 3 bp to 5 bp furtherly inserted into the 5′-terminal is added.

In order to solve problems described above, the present invention provides a dumbbell structure oligonucleotide represented by the following general formula:

5′-Aₚ-B_{q}-Cᵣ-3′ Formula:

Herein, A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and p, q and r each represent the number of nucleotides.

p preferably comprises 3 to 5 nucleotides.

q preferably comprises 3 to 5 nucleotides.

r preferably comprises 18 to 30 nucleotides.

Tₘ of the 5′-low Tₘ specificity site is preferably lower than Tₘ of the 3′-high Tₘ specificity site.

Tₘ of the cleavage site is preferably lower than Tₘ of the 5′-low Tₘ specificity site and Tₘ of the 3′-high Tₘ specificity site.

Tₘ of the 5′-low Tₘ specificity site is preferably 10° C. to 30° C.

Tₘ of the cleavage site is preferably 3° C. to 10° C.

Tₘ of the 3′-high Tₘ specificity site is preferably 50° C. to 65° C.

The universal base is preferably one selected from the group consisting of deoxyinosine, inosine, 7-diaza-2′-deoxyinosine, 2-aza-2′-deoxyinosine, 2′-OMe inosine, 2′-inosine, and a combination thereof.

The dumbbell structure oligonucleotide is preferably one selected from the group consisting of sequence number 1 to sequence number 35.

The present invention also provides a nucleic acid amplification primer comprising a dumbbell structure oligonucleotide represented by the following general formula:

5′-Aₚ-B_{q}-Cᵣ-3′ Formula:

Herein, A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and p, q and r each represent the number of nucleotides.

p preferably comprises 3 to 5 nucleotides.

q preferably comprises 3 to 5 nucleotides.

r preferably comprises 18 to 30 nucleotides.

Tₘ of the 5′-low Tₙ specificity site is preferably lower than Tₘ of the 3′-high Tₘ specificity site.

Tₘ of the cleavage site is preferably lower than Tₘ of the 5′-low Tₘ specificity site and Tₘ of the 3′-high Tₘ specificity site.

Tₘ of the 5′-low Tₘ specificity site is preferably 10° C. to 30° C.

Tₘ of the cleavage site is preferably 3° C. to 10° C.

Tₘ of the 3′-high Tₘ specificity site is preferably 50° C. to 65° C.

The universal base is preferably one selected from the group consisting of deoxyinosine, inosine, 7-diaza-2′-deoxyinosine, 2-aza-2′-deoxyinosine, 2′-OMe inosine, 2′-inosine, and a combination thereof.

The dumbbell structure oligonucleotide is preferably one selected from the group consisting of sequence number 1 to sequence number 35.

The present invention also provides a method for amplifying a nucleic acid by performing a polymerase chain reaction from a mixture comprising a template, a primer and a polymerase, using a nucleic acid amplification primer comprising a dumbbell structure oligonucleotide represented by the following general formula:

5′-Aₚ-B_{q}-Cᵣ-3′ Formula:

Herein, A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and p, q and r each represent the number of nucleotides.

p preferably comprises 3 to 5 nucleotides.

q preferably comprises 3 to 5 nucleotides.

r preferably comprises 18 to 30 nucleotides.

Tₘ of the 5′-low Tₘ specificity site is preferably lower than Tₘ of the 3′-high Tₘ specificity site.

Tₘ of the cleavage site is preferably lower than Tₘ of the 5′-low Tₘ specificity site and Tₘ of the 3′-high Tₘ specificity site.

Tₘ of the 5′-low Tₘ specificity site is preferably 10° C. to 30° C.

Tₘ of the cleavage site is preferably 3° C. to 10° C.

Tₘ of the 3′-high Tₘ specificity site is preferably 50° C. to 65° C.

The universal base is preferably one selected from the group consisting of deoxyinosine, inosine, 7-diaza-2′-deoxyinosine, 2-aza-2′-deoxyinosine, 2′-OMe inosine, 2′-inosine, and a combination thereof.

The dumbbell structure oligonucleotide is preferably used as nucleic amplification primer one selected from the group consisting of sequence number 1 to sequence number 35.

The nucleic acid amplification method is preferably a multiple polymerase chain reaction using two or more templates.

The present invention uses the dumbbell structure oligonucleotide (DSO) obtained by adding the arbitrary base complementarily designed to complementarily connect the 3′-terminal oligonucleotide and the 5′-terminal oligonucleotide, the 3′-terminal template-dependent specific base sequence, and the universal base pair connecting the two base sequences before binding the template at each first cycle upon the polymerase chain reaction (PCR) to suppress unintended amplification products at the room temperature, and as a result, it is possible to innovate the gene amplification method efficiently by increasing sensitivity and specificity according to the reduction of non-specific amplification products. Using the gene amplification method of the present invention, it is possible not only to amplify a large number of genes by only a single polymerase chain reaction, but also to more easily detect single nucleotide polymorphism analysis, thereby contributing to advancement of research and development of gene related fields.

### BRIEF DESCRIPTION OF THE DRAWINGS

FIG. 1 is a view illustrating structural features of a primer used in a simultaneous multi-gene amplification method,

FIG. 2 is a schematic view illustrating that the amplification of a PCR product as a template is dominant as compared with the initial template-based amplification from the third cycle during PCR,

FIG. 3 illustrates the principle of the dumbbell structure oligonucleotides of the present invention in a target-dependent extension reaction. (a) shows that the amplification may not take place due to the high hybridization specificity of the dumbbell structure oligonucleotide under high stringency conditions, and (b) shows that a successful extension reaction of the dumbbell structure oligonucleotide occurs,

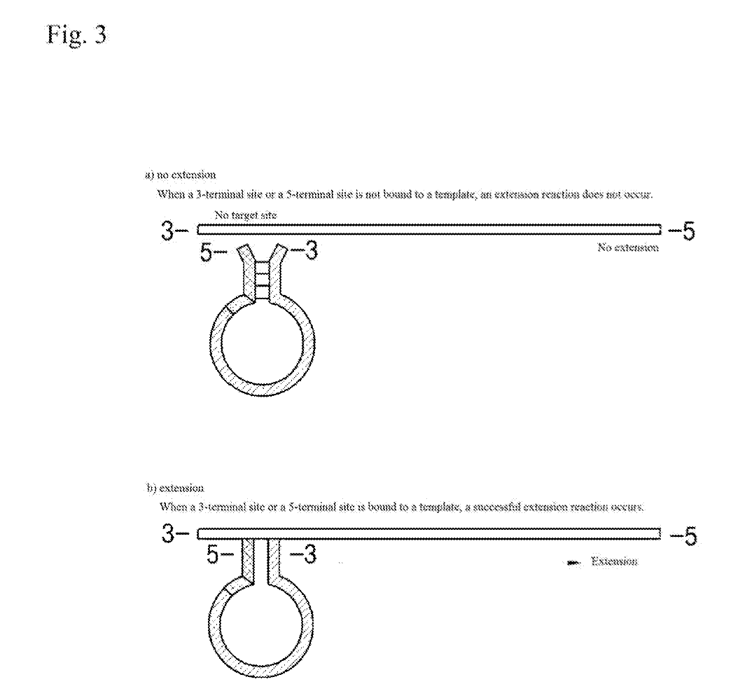

FIG. 4 is an electrophoresis image of a sexual transmitted disease causative microorganism amplified using a gene amplification method,

FIG. 5 illustrates a result of SNP readings of the MTHFR gene C677T by the allele-specific polymerase chain reaction with a dumbbell structure oligonucleotide,

FIG. 6 illustrates a result of SNPs readings of the BRAF gene V600E by the allele-specific polymerase chain reaction with the dumbbell structure oligonucleotide, and

FIG. 7 illustrates a result of SNPs readings of the APC gene by the allele-specific polymerase chain reaction with the dumbbell structure oligonucleotide.

### DETAILED DESCRIPTION OF EXEMPLARY EMBODIMENTS

Hereinafter, the present invention is described in detail.

In order to develop a method for amplifying a plurality of genes by a single polymerase chain reaction, the inventors of the present invention conducted various studies, and was noted that if each of the genes used as a template and each of the primers capable of complementarily binding thereto could be specifically bound, a large number of genes could be amplified by one polymerase chain reaction, and it was intended to increase the specific selectivity of the primer to the template gene.

Therefore, as a preferable embodiment of the present invention, there is provided a method for producing a nucleic acid molecule by a template-dependent extension reaction using a dumbbell structure oligonucleotide.

In another preferable embodiment of the present invention, there is provided a method for selectively amplifying a target nucleic acid sequence in a single DNA or a mixture of nucleic acids.

In still another preferable embodiment of the present invention, there is provided a method for amplifying two or more target nucleotide sequences simultaneously using two or more primer pairs in the same reaction.

In still another preferable embodiment of the present invention, there is provided a method for detecting a nucleic acid molecule having genetic diversity by a template-dependent extension reaction.

In still another preferable embodiment of the present invention, there is provided a dumbbell structure oligonucleotide for producing a nucleic acid molecule by a template-dependent extension reaction.

In further another preferable embodiment of the present invention, there is provided a method for increasing the annealing specificity of an oligonucleotide.

The various embodiments of the present invention as described above, will become more apparent from the following detailed description of the invention, claims and drawings.

The present invention relates to a dumbbell structure oligonucleotides and various methods using the same. The dumbbell structure oligonucleotide of the present invention allows the primers or probes to anneal to the target nucleic acid with increased specificity, thereby greatly increasing the specificity of nucleic acid amplification (especially PCR).

Thus, the present invention provides a dumbbell structure oligonucleotide represented by the following general formula:

5′-Aₚ-B_{q}-Cᵣ-3′ Formula:

Herein, A represents a 5′-low Tₘ specificity site including a nucleotide having a base sequence complementary to the consecutive nucleotide sequence of the 3′-terminal, B represents a cleavage site including a nucleotide having a universal base, C represents a 3′-high Tₘ specificity site including the nucleotide having the base sequence complementary to the specific consecutive base sequence of the template nucleic acid, and p, q and r each represent the number of nucleotides.

p preferably comprises 3 to 5 nucleotides.

q preferably comprises 3 to 5 nucleotides.

r preferably comprises 18 to 30 nucleotides.

Tₘ of the 5′-low Tₘ specificity site is preferably lower than Tₘ of the 3′-high Tₘ specificity site.

Tₘ of the cleavage site is preferably lower than Tₘ of the 5′-low Tₘ specificity site and Tₘ of the 3′-high Tₘ specificity site.

Tₘ of the 5′-low Tₘ specificity site is preferably 10° C. to 30° C.

Tₘ of the cleavage site is preferably 3° C. to 10° C.

Tₘ of the 3′-high Tₘ specificity site is preferably 50° C. to 65° C.

The universal base is preferably one selected from the group consisting of deoxyinosine, inosine, 7-diaza-2′-deoxyinosine, 2-aza-2′-deoxyinosine, 2′-OMe inosine, 2′-inosine, and a combination thereof.

The dumbbell structure oligonucleotide is preferably one selected from the group consisting of sequence number 1 to sequence number 35.

The present invention also provides a nucleic acid amplification primer comprising a dumbbell structure oligonucleotide represented by the following general formula:

5′-Aₚ-B_{q}-Cᵣ-3′ Formula:

Herein, A represents a 5′-low Tₘ specificity site including a nucleotide having the base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and the p, q and r each represent the number of nucleotides.

p preferably comprises 3 to 5 nucleotides.

q preferably comprises 3 to 5 nucleotides.

r preferably comprises 18 to 30 nucleotides.

Tₘ of the 5′-low Tₘ specificity site is preferably lower than Tₘ of the 3′-high Tₘ specificity site.

Tₘ of the cleavage site is preferably lower than Tₘ of the 5′-low Tₘ specificity site and Tₘ of the 3′-high Tₘ specificity site.

Tₘ of the 5′-low Tₘ specificity site is preferably 10° C. to 30° C.

Tₘ of the cleavage site is preferably 3° C. to 10° C.

Tₘ of the 3′-high Tₘ specificity site is preferably 50° C. to 65° C.

The universal base is preferably one selected from the group consisting of deoxyinosine, inosine, 7-diaza-2′-deoxyinosine, 2-aza-2′-deoxyinosine, 2′-OMe inosine, 2′-inosine, and a combination thereof.

The dumbbell structure oligonucleotide is preferably one selected from the group consisting of sequence number 1 to sequence number 35.

The present invention also provides a method for amplifying a nucleic acid by performing a polymerase chain reaction from a mixture comprising a template, a primer and a polymerase, using a nucleic acid amplification primer comprising a dumbbell structure oligonucleotide represented by the following general formula:

5′-Aₚ-B_{q}-Cᵣ-3′ Formula:

Herein, A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and the p, q and r each represent the number of nucleotides.

p preferably comprises 3 to 5 nucleotides.

q preferably comprises 3 to 5 nucleotides.

r preferably comprises 18 to 30 nucleotides.

Tₘ of the 5′-low Tₘ specificity site is preferably lower than Tₘ of the 3′-high Tₘ specificity site.

Tₘ of the cleavage site is preferably lower than Tₘ of the 5′-low Tₘ specificity site and Tₘ of the 3′-high Tₘ specificity site.

Tₘ of the 5′-low Tₘ specificity site is preferably 10° C. to 30° C.

Tₘ of the cleavage site is preferably 3° C. to 10° C.

Tₘ of the 3′-high Tₘ specificity site is preferably 50° C. to 65° C.

The universal base is preferably one selected from the group consisting of deoxyinosine, inosine, 7-diaza-2′-deoxyinosine, 2-aza-2′-deoxyinosine, 2′-OMe inosine, 2′-inosine, and a combination thereof.

The dumbbell structure oligonucleotide is preferably used as nucleic amplification primer one selected from the group consisting of sequence number 1 to sequence number 35.

The nucleic acid amplification method is preferably a multiple polymerase chain reaction using two or more templates.

According to one aspect of the present invention, the present invention provides a dumbbell structure oligonucleotide represented by the following general formula and for synthesizing a nucleic acid molecule by a template-dependent extension reaction.

5′-Aₚ-B_{q}-Cᵣ-3′ Formula:

A represents a base sequence substantially and complementarily binding a consecutive nucleotide sequence from 3′-terminal of the formula as described above, B represents a cleavage site including a universal base, C substantially represents a complementary base sequence regarding one site of a hybridized template nucleic acid, and p, q and r represent the number of nucleotides. A, B, and C are deoxyribonucleotides or ribonucleotides, and the cleavage site has the lowest Tₘ among the three sites of A, B, and C, the cleavage site forms a non-base pair hairpin structure under the condition that A and B bind to the template nucleic acid so that in terms of the binding specificity to the template nucleic acid, A is separated from B, and the binding specificity of the oligonucleotide is determined by both A and B to increase the specific selectivity of the primer to the template gene.

The dumbbell structure oligonucleotide of the present invention is very useful in a variety of fields, such as the Miller, H. I. method (WO 89/06700) and Davey, C. et al. (EP 329,822), related nucleic acid amplification methods, e.g., ligase chain reaction (LCR, Wu, D Y et al., Genomics 4 560 (1989)), polymerase ligase chain reaction (Barany, PCR Methods and Appl., 1: 5-16 (1991)), gap-LCR (WO 90/01069), restorative chain reaction (EP 439,182), 3SR (Kwoh et al., PNAS, USA, 86: 1173 (1989)), and NASBA (U.S. Pat. No. 5,130,238), primer extension-related techniques e.g., cycle sequencing (Kretz et al. (1998) Science 281: 363-365), PCR sequencing method (PCR Methods Appl. 3: S107-SI 12), and pirosequencing (Ronaghi et al., (1996) Anal. Biochem., 24284-89 and Science 281:363-365), and hybridization-related techniques such as detection of target nucleotide sequences using oligonucleotide microarrays.

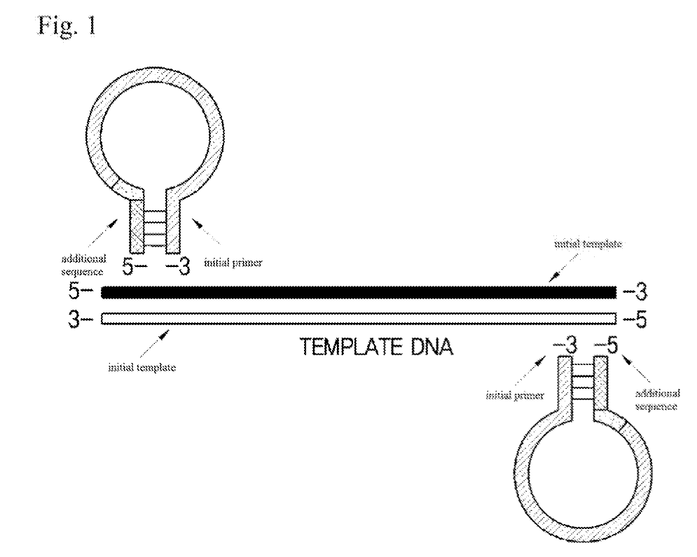

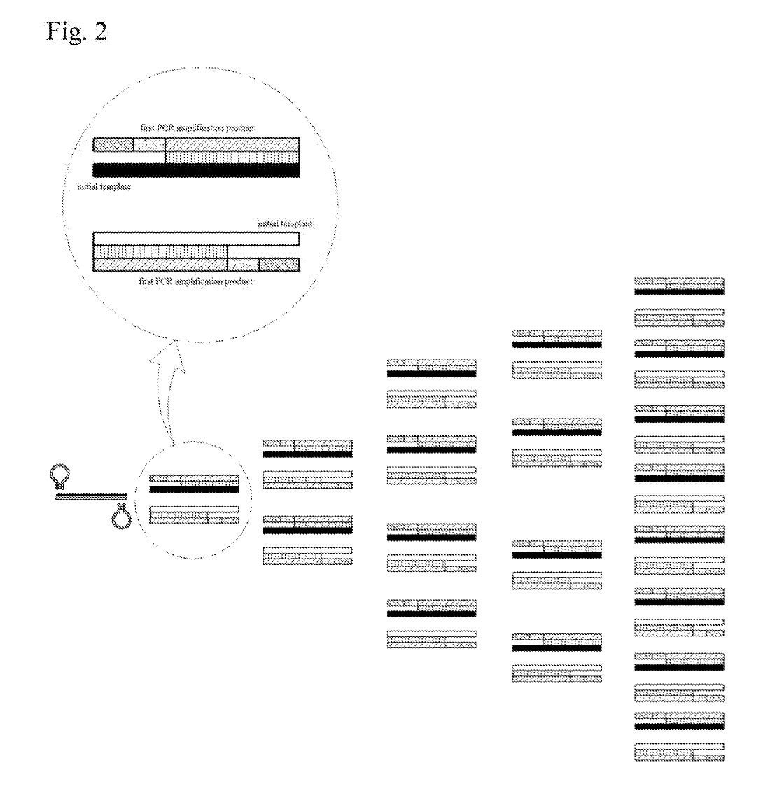

FIG. 1 is a view illustrating structural features of a primer used in a simultaneous multi-gene amplification method. As illustrated in FIG. 1, when preparing the primer of the gene to be amplified, the arbitrary base sequence of 3 bp to 5 bp, complementary to the 3′-terminal is added to the 5′-terminal, so that nonspecific binding is inhibited by not complementarily binding to the template base sequence upon performing each first cycle, the universal base pairs of 3 bp to 5 bp are substituted to form a bulge at a center when hybridizing with the template gene in the polymerase chain reaction, and the base sequence of 3 bp to 5 bp at the 5′-terminal site is substituted with the base sequence capable of complementarily binding with the 3′-terminal site, and the base sequence of a 3′-terminal site is constructed in a primer comprising the dumbbell structure oligonucleotide having a form capable of complementarily binding with the gene to be amplified. When the PCR amplification is performed using the constructed primer, although the annealing temperature is changed, the template gene is normally amplified. On the other hand, when performing the PCR amplification using the conventional primer under the condition that the annealing temperature is changed, as the annealing temperature is increased, the amplification rate is lowered, and the template gene is not normally amplified (See FIG. 2). When the PCR amplification is carried out using the DSO primer of the present invention, specific selectivity of the primer to the template gene is increased, and thus if a plurality of template genes and corresponding primers are mixed to perform the single polymerase chain reaction, each template gene may be amplified normally. The method by which a plurality of template genes may be amplified by a single polymerase chain reaction using such DSO primer is called “DIGPlex™.”

Therefore, a simultaneous multi-gene amplification method comprises: (i) a step of selecting each site to be amplified from 2 to 30 target genes; (ii) a step of determining the arbitrary base sequence of the 5′-terminal complementarily binding to the base sequence of the 3′-terminal of the each selected site and constructing a sense primer to which the universal base pairs of 3 bp to 5 bp located at a center of the determined base sequence is added; (iii) a step of determining the base sequence complementarily binding to the base sequence of the 3′-terminal of the each selected site and constructing an antisense primer to which the universal base pairs of ˜5 bp located at the center of the determined base sequence is added; (iv) a step of mixing the above 2 to 30 target genes together with the constructed 2 to 30 sense primers and antisense primers respectively corresponding to the target genes and performing the single polymerase chain reaction using the mixture; and (v) a step of identifying the amplification product obtained by the polymerase chain reaction. Herein, the temperature and time conditions in the PCR reaction are not particularly limited. Further, confirmation of the obtained amplification products is not particularly limited.

Using the simultaneous multi-gene amplification method of the present invention, a large number of genes may be amplified by only single polymerase chain reaction, and the single base polymorphism analysis may be easily implemented, thereby contributing to advances in the research and development of gene related fields.

Hereinafter, the present invention will be described in more detail with reference to embodiments for confirming the presence or absence of causative bacteria of infectious diseases, and single nucleotide polymorphisms analyses of MTHFR gene causing cardiovascular disease, BRAF gene responsible for thyroid papillary cancer, and APC gene related to colorectal cancer. It is understood by those skilled in the an that these embodiments are only for describing the present invention in more detail and that the scope of the present invention is not limited by these embodiments in accordance with the point of the present invention.

### EMBODIMENTS

### Embodiment 1: Amplification of a Sexual Transmitted Disease Specific Gene

DNAs extracted from samples obtained from 20 patients suspected of having sexual transmitted disease were mixed with 2 μl of a 10× polymerase chain reaction buffer solution (750 mM Tris-HCl (pH 9.0), 20 mM MgCl₂, 500 mM KCl, 200 mM (NH₄)₂SO₄), 2 μl of 2.5 mM dNTP mixture (2.5 mM dATP, 2.5 mM dGTP, 2.5 mM dTTP, 2.5 mM dCTP), 2.0 units of Taq polymerase (Biotools, Spain), and 1 μl of DS primer (0.5 μM) having base sequence of sequence list 1 to 26, 36, and 36, then type III purified water was added to the mixture to be titrated to 20 μl, and then polymerase chain reaction was performed (94° C. for 10 minutes, 94° C. for 30 seconds, 65° C. for 60 seconds, 72° C. for 60, 35 cycles) to obtain an amplification product. Here, the base sequences of the respective DSO primers used are shown in Table 1 below. For reference, the letter “N” in the base sequence shown in sequence number 1 to sequence number 37 in the sequence listing attached to the present specification means “Inosine, I” as shown in Tables 1 and 2 below.

TABLE 1

| Disease | | | target |
|---|---|---|---|
| type 1) | 5′-terminal primer | 3′-terminal primer | gene |
| CT | CGAGG III GACTACCCAA | CGCAT III CAAGCCAA | ompA |
| | ACCTTCAACGACACCTCG | GACCGCAAGTGAATAATGCG | |
| NG | CCAAA III TTACAGACTG | CAAGC III GTCGACTGCA | porA |
| | GCGGCGGTTTCGTTTTGG | CACCCGAACAGCTTG | |
| TV | CAGCT III CCTCGATGTC | GTCAG III GAGCTTACGA | btub1 |
| | ATCCGTAAGGAAGCTG | AGGTCGGAGTTGAGCTGAC | |
| MG | CATCT III TGCCAATCCT | GACCA III CCTAGCTCCT | gyrA |
| | AAGATAAATTCCAAACCAGA | TATAAGCTTGAACTGCTGGTC | |
| | AGAGATG | | |
| UU | GATAT III CGCCCGTCAA | GCTTT III CTGAGTTTCCTCA | gap |
| | ACTATGGGAGCTGGTAATATC | TTCGGAGATCAACGGATTAAAGC | |
| MH | CATGT III GGATGAACGC | GACTG III CATCGCTTTCTGA | gap |
| | TGGCTGTGTGCCTAATACATG | CAAGGTACCGTCAGTC | |
| GV | CACTC III CATCGAATCT | GCGGT III GATATACGTGGTG | 16s |
| | TTGAACGCACATTGCG | GACGTTACCGC | |
| CA | CGCAA III CATCGAATCT | GCGGT III GATATACGTGGTG | phr1 |
| | TTGAACGCACATTGCG | GACGTTACCGC | |
| HSV1 | CCAGG III GT CAACGAC | GAGAT III CAGACGGAGCCGT | g1yC |
| | CATATTCACGCCT GG | TGGTGATAAGATCTC | |
| HSV2 | CTTGA III GATGTTTGCT | CATGA III CCGTCGGGGACTG | g1yC |
| | TGGTCGTTCCTGGTCCTCAAG | AACGTCTCATG | |
| UP | GCAAG III GTCCATTTCA | CACCTIII TGGAGCATTAATTT | gap |
| | ACAAGCACGCAAACTTGC | GGCTATCATCTTTTTGAATAGGTG | |
| TP | GTATG III GTGCGTACTCG | CGAGG III GGGCTGCAATTCTT | p47 |
| | GAGCTTGCAGAGAAGACATAC | TGTTCTTCGAGTTTTCGTGCCTCG | |
| IC | GGACA III CACAAGTATCA | GGATA III GCAGAATCCAGATG | GAPDH |
| | CTAAGCTCGCTTTCTTGCTGT | CTCAAGGCCCTTCATAATATCC | |
| | CC | | |

1) Disease type
TP: Treponema pallidum
MG: Myroplasma Genitalium
NG: Neisseria gonorrhoeae
MR: Mycop asma hominis
UU: Ureaplasma urealyticum
GV: Gardenerella vagnialis
CT: Chiamydia trachomatis
HSV2: Herpes Simplex Virus
2CA: Candida albicans
HSV1: Herpes Simplex Virus
1 UP: Ureaplasma parvum
IC: GAPDH

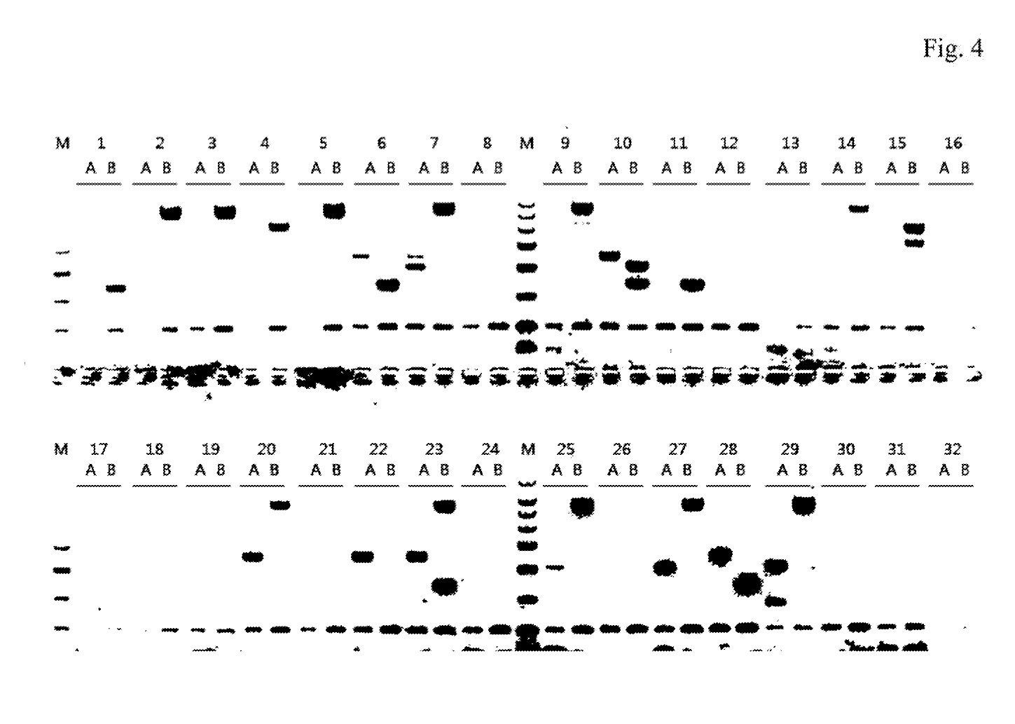

Then, the reaction product of the polymerase chain reaction was electrophoresed on 2.0% agarose gel (see FIG. 4). FIG. 4 is an electrophoresis image of 12 sexual transmitted disease causative microorganisms amplified by the polymerase chain reaction. In FIG. 4, No. 1 shows an image obtained by amplifying a clinical sample infected CA, No. 2 shows an image obtained by amplifying a clinical sample infected with UP and GV, No. 3 shows an image obtained by amplifying a clinical sample infected with GV, No. 4 shows an image obtained by amplifying a clinical sample infected with CT, No. 5 shows an image obtained by amplifying a clinical sample infected with GV, No. 6 shows an image obtained by amplifying a clinical sample infected with UP, and CA, No. 7 represents an image obtained by amplifying a clinical sample infected with MH, UP, or GV, No. 8 represents an image obtained by amplifying a negative sample, No. 9 represents an image obtained by amplifying a clinical sample infected with GV or CT, No. 10 shows an image obtained by amplifying a clinical sample infected with UP, HSV2, and CA, No. 11 shows an image obtained by amplifying a clinical sample infected with CA, Nos. 12 and 13 show an image obtained by amplifying a negative sample, No. 14 shows an image obtained by amplifying a clinical sample infected with GV and CT, No. 15 shows an image obtained by amplifying a clinical sample infected with CT and TV, Nos. 16, 17, 18, and 19 show an image obtained by amplifying a negative sample, No. 20 represents an image obtained by amplifying a clinical sample infected with UP and GV, No. 21 represents an image obtained by amplifying a negative sample, No. 22 represents an image obtained by amplifying a clinical sample infected with UP, No. 23 represents an image obtained by amplifying a clinical sample infected with UP, GV, and CA, No. 24 represents an image obtained by amplifying a negative sample, No. 25 represents an image obtained by amplifying a clinical sample infected with MH and GV, No. 26 shows an image obtained by amplifying a clinical sample infected with UP, No. 27 shows an image obtained by amplifying a clinical sample infected with MH and GV, No. 28 shows an image obtained by amplifying a clinical sample infected with UP and CA, No. 29 shows an image obtained by amplifying a clinical sample infected with MH, UU and GV. No. 30 shows an image obtained by amplifying a clinical sample infected with CA, No. 31 shows an image obtained by amplifying a negative sample, and No. 32 shows an image obtained by amplifying a negative control. As seen from the above results, it was confirmed that multiple genes may be amplified by a single polymerase chain reaction when using the simultaneous multi-gene amplification method using the DSO primer of the present invention.

### Embodiment 2: Single Base Polymorphism Analysis of MTHFR Gene, BRAF Gene, and APC Gene Using DSO Primer

To amplify commercially available human MTHFR, BRAF, and APC genes (wild type, hetero type, homo type), a human genomic DNA (Invitrogen Inc., USA) was used as a template, genes were amplified using a normal primer having the following base sequence, and then confirmation of the results by the restriction enzyme treatment and the polymerase chain reaction of the present invention were performed to confirm amplification products.

TABLE 2

| | 5′-terminal | | |
|---|---|---|---|
| type | primer | 3′-terminal primer | gene |
| WILD | CATCTTIIITGCTGT | CCGATIIIGCGTGATGATGA | MTHFR |
| | TGGAAGGTGCAAGAT | AATCGG | |
| | (sequence | (sequence number 28) | |
| MUTANT | number 27) | TCGATIIIGCGTGATGATGA | |
| | | AATCGA | |
| | | (sequence number 29) | |
| WILD | CAATGIIIGAATATC | TGAAAIIICACTCCATCGAG | BRAF |
| | TGGGCCTACATTG | ATTTCA | |
| | (sequence | (sequence number 31) | |
| MUTANT | number 30) | AGAAAIIICACTCCATCGAG | |
| | | ATTTCT | |
| | | (sequence number 32) | |
| WILD | GAGGTIIICCACACA | AGTTT III TTATGAGAAA | APC |
| | GAACTAACCTC | AGCAAACT | |
| | (sequence | (sequence number 34) | |
| MUTANT | number 33) | GGTTT IIITTATGAGAAAA | |
| | | GCAAACC | |
| | | (sequence number 35) | |

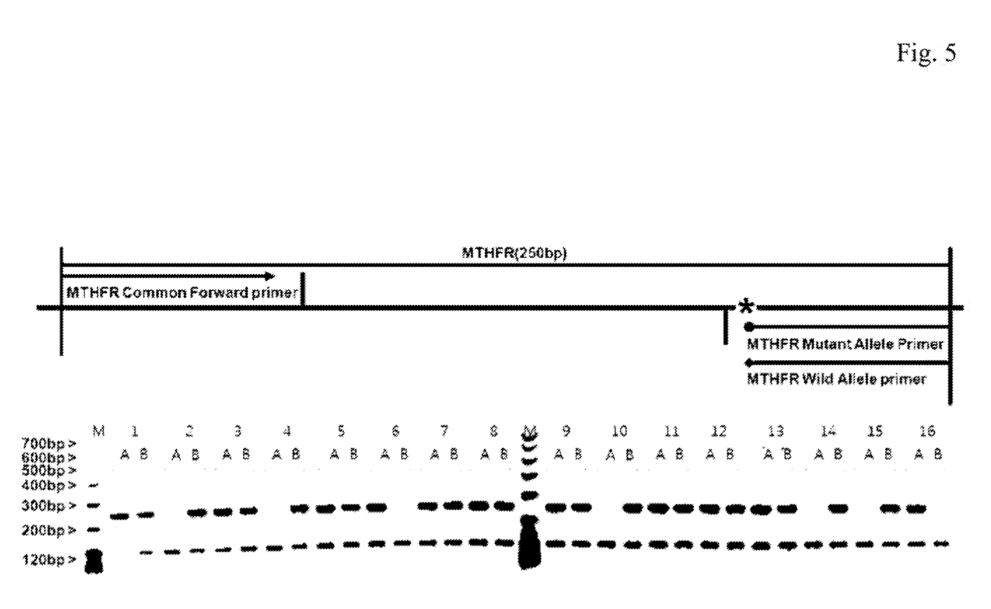

2 μl (50 ng/μl) of genomic DNA extracted from human blood, 2 μl of 10× polymerase chain reaction buffer solution (750 mM Tris-HCl (pH 9.0), 20 mM MgCl₂, 500 mM KCl, 200 mM (NH₄)₂SO₄), 2 μl of 2.5 mM dNTP mixture (2.5 mM dATP, 2.5 mM dGTP, 2.5 mM dTTP, 2.5 mM dCTP), 1.5 unit of Taq polymerase (Biotools, Spain), and 1 μl of each mixture of the DSO primers (0.5 μM) were mixed, and type III purified water was added to the mixture to be titrated to 20 μl, and then polymerase chain reaction was performed. Herein, polymerase chain reaction was performed for 35 cycles under conditions of 94° C. for 10 minutes, 94° C. for 30 seconds, annealing for 60 seconds, and 72° C. for 60 seconds, and each annealing temperature was different in 60° C., 55° C., and 62° C. After the reaction was completed, the amplified fragment was electrophoresed on a 2% (w/v) agarose gel (see FIGS. 5, 6 and 7).

FIG. 5 shows the results of performing the allele-specific polymerase chain reaction analysis on 16 clinical samples confirmed by the polymerase chain reaction-restriction fragment length polymorphism analysis as described above in order to confirm whether there is the mutation of the MTHFR gene closely related to cardiovascular disease. As an image, M is a size marker that confirms amplification product size. In this analysis, clinical samples 1, 3, 5, 7, 8, 9, 11, 12, and 13 were determined to be samples with a high homocysteine concentration and were found to have both a wild type and a mutant type. Clinical samples 2, 4, 10, 14, and 15 were determined to be samples with cardiovascular disease and were found to have only the mutant type. Clinical samples 6 and 16 were determined to be a normal person and were found to have only the wild type.

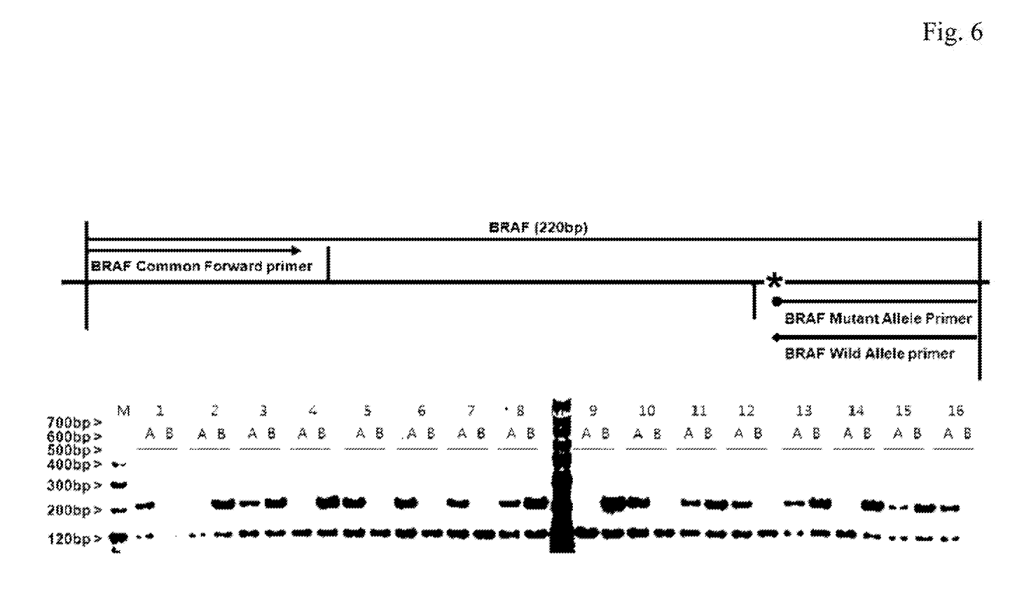

FIG. 6 shows the results of performing the allele-specific polymerase chain reaction analysis on 16 clinical samples confirmed by the polymerase chain reaction-restriction fragment length polymorphism analysis as described above in order to confirm whether there is the mutation of the BRAF gene closely related to thyroid cancer. As an image, M is a size marker that confirms amplification product size. In this analysis, clinical samples 1, 5, 6, 7, 10, 12, and 16 were determined to be a normal person and were found to have only the wild type. Clinical samples 3, 8, 11, 13, and 15 were determined to be clinical samples of patients with abnormal thyroid function and were found to have both the wild type and the mutant type. Clinical samples 2, 4, 9, and 14 were determined to be patients with thyroid cancer and were found to have only the mutant type.

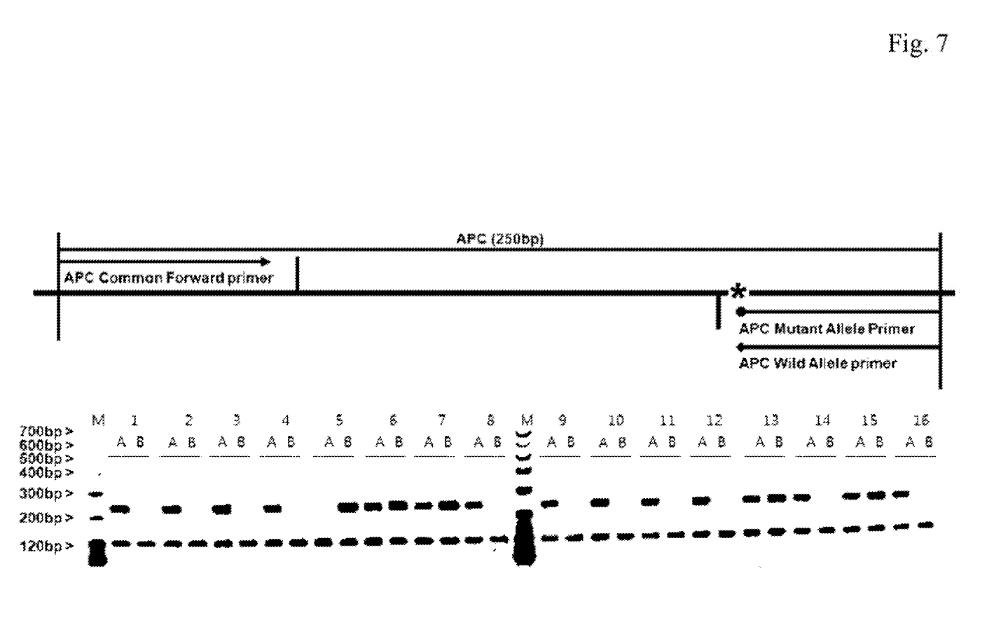

FIG. 7 shows the results of performing the allele-specific polymerase chain reaction analysis on 16 clinical samples confirmed by the polymerase chain reaction-restriction fragment length polymorphism analysis as described above in order to confirm whether there is the mutation of the APC gene closely related to familial polyposis (colorectal cancer). As an image, M is a size marker that confirms amplification product size. In this analysis, clinical samples 1 1, 2, 3, 4, 8, 9, 10, 11, 12, 14 and 16 were determined to be a normal person and were found to have only the wild type. Clinical samples 6, 7, 13 and 15 were conformed to have polyposis as results obtained by colonoscopy and were found to have both the wild type and the mutant type. Clinical sample 5 was determined to be a zero-stage intraepithelial carcinoma patient and was found to have only the mutant type.

What is claimed is:

1. A dumbbell structure oligonucleotide represented by the following general formula:
5′-Aₚ-B_{q}-Cᵣ-3′ Formula:
wherein A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and p, q and r each represent the number of nucleotides.

2. The dumbbell structure oligonucleotide of claim 1, wherein p comprises 3 to 5 nucleotides.

3. The dumbbell structure oligonucleotide of claim 1, wherein q comprises 3 to 5 nucleotides.

4. The dumbbell structure oligonucleotide of claim 1, wherein r comprises 18 to 30 nucleotides.

5. The dumbbell structure oligonucleotide of claim 1, wherein Tₘ of the 5′-low Tₘ specificity site is lower than Tₘ, of the 3′-high Tₘ specificity site.

6. The dumbbell structure oligonucleotide of claim 1, wherein Tₘ of the cleavage site is lower than Tₘ of the 5′-low Tₘ specificity site and Tₘ of the 3′-high Tₘ specificity site.

7. The dumbbell structure oligonucleotide of claim 1, wherein Tₘ of the 5′-low Tₘ specificity site is 10° C. to 30° C.

8. The dumbbell structure oligonucleotide of claim 1, wherein Tₘ of the cleavage site is 3° C. to 10° C.

9. The dumbbell structure oligonucleotide of claim 1, wherein Tₘ of the 3′-high Tₘ specificity site is 50° C. to 65° C.

10. The dumbbell structure oligonucleotide of claim 1, wherein the universal base is one selected from the group consisting of deoxyinosine, inosine, 7-diaza-2′-deoxyinosine, 2-aza-2′-deoxyinosine, 2′-OMe inosine, 2′-inosine, and a combination thereof.

11. The dumbbell structure oligonucleotide of claim 1, wherein the dumbbell structure oligonucleotide is one selected from the group consisting of sequence number 1 to sequence number 35.

12. A nucleic acid amplification primer comprising a dumbbell structure oligonucleotide represented by the following general formula:
5′-Aₚ-B_{q}-Cᵣ-3′ Formula:
wherein, A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and p, q and r each represent the number of nucleotides.

13. The nucleic acid amplification primer of claim 12, wherein p comprises 3 to 5 nucleotides.

14. The nucleic acid amplification primer of claim 12, wherein q comprises 3 to 5 nucleotides.

15. The nucleic acid amplification primer of claim 12, wherein r comprises 18 to 30 nucleotides.

16. The nucleic acid amplification primer of claim 12, wherein Tₘ of the 5′-low Tₘ specificity site is lower than Tₘ of the 3′-high Tₘ specificity site.

17. The nucleic acid amplification primer of claim 12, wherein Tₘ of the cleavage site is lower than Tₘ of the 5′-low Tₘ specificity site and Tₘ of the 3′-high Tₘ specificity site.

18. The nucleic acid amplification primer of claim 12, wherein Tₘ of the 5′-low Tₘ specificity site is 10° C. to 30° C.

19. The nucleic acid amplification primer of claim 12, wherein Tₘ of the cleavage site is 3° C. to 10° C.

20. The nucleic acid amplification primer of claim 12, wherein Tₘ of the 3′-high Tₘ specificity site is 50° C. to 65° C.

21. The nucleic acid amplification primer of claim 12, wherein the universal base is one selected from the group consisting of deoxyinosine, inosine, 7-diaza-2′-deoxyinosine, 2-aza-2′-deoxyinosine, 2′-OMe inosine, 2′-inosine, and a combination thereof.

22. The nucleic acid amplification primer of claim 12, wherein the dumbbell structure oligonucleotide is one selected from the group consisting of sequence number 1 to sequence number 35.

23. A method for amplifying a nucleic acid by performing a polymerase chain reaction from a mixture comprising a template, a primer, and a polymerase, using a nucleic acid amplification primer comprising a dumbbell structure oligonucleotide represented by the following general formula:
5′-Aₚ-B_{q}-Cᵣ-3′ Formula:
wherein, A represents a 5′-low Tₘ specificity site including a nucleotide having a base sequence complementary to the consecutive nucleotide sequence of a 3′-terminal, B represents a cleavage site including a nucleotide having a universal base, C represents a 3′-high Tₘ specificity site including a nucleotide having a base sequence complementary to a specific consecutive base sequence of a template nucleic acid, and p, q and r each represent the number of nucleotides.

24. The method for amplifying a nucleic acid of claim 23, wherein p comprises 3 to 5 nucleotides.

25. The method for amplifying a nucleic acid of claim 23, wherein q comprises 3 to 5 nucleotides.

26. The method for amplifying a nucleic acid of claim 23, wherein r comprises 18 to 30 nucleotides.

27. The method for amplifying a nucleic acid of claim 23, wherein Tₘ of the 5′-low Tₘ specificity site is lower than Tₘ of the 3′-high Tₘ specificity site.

28. The method for amplifying a nucleic acid of claim 23, wherein Tₘ of the cleavage site is lower than Tₘ of the 5′-low Tₘ specificity site and Tₘ of the 3′-high Tₘ specificity site.

29. The method for amplifying a nucleic acid of claim 23, wherein Tₘ of the 5′-low Tₘ specificity site is 10° C. to 30° C.

30. The method for amplifying a nucleic acid of claim 23, wherein Tₘ of the cleavage site is 3° C. to 10° C.

31. The method for amplifying a nucleic acid of claim 23, wherein Tₘ of the 3′-high Tₘ specificity site is 50° C. to 65° C.

32. The method for amplifying a nucleic acid of claim 23, wherein the universal base is one selected from the group consisting of deoxyinosine, inosine, 7-diaza-2′-deoxyinosine, 2-aza-2′-deoxyinosine, 2′-OMe inosine, 2′-inosine, and a combination thereof.

33. The method for amplifying a nucleic acid of claim 23, wherein the dumbbell structure oligonucleotide is preferably used as nucleic amplification primer one selected from the group consisting of sequence number 1 to sequence number 35.

34. The method for amplifying a nucleic acid of claim 23, wherein the nucleic acid amplification method is a multiple polymerase chain reaction using two or more templates.
